(19) United States
(12) Patent Application Publication
(10) Pub. No.: US 2022/0218102 A1
(43) Pub. Date: Jul. 14, 2022

(54) RETRACTABLE SELF-SANITIZING DIVIDER ASSEMBLY

(71) Applicant: Charles Isgar, Scottsdale, AZ (US)

(72) Inventor: Charles Isgar, Scottsdale, AZ (US)

(21) Appl. No.: 17/713,061

(22) Filed: Apr. 4, 2022

Related U.S. Application Data
(63) Continuation-in-part of application No. 17/201,861, filed on Mar. 15, 2021, now Pat. No. 11,291,299.
(60) Provisional application No. 63/022,419, filed on May 8, 2020.

(51) Int. Cl. A47B 21/02; A61L 2/22

(52) U.S. Cl. CPC A47B 21/02; A61L 2/22

(57) ABSTRACT

A self-sanitizing divider assembly is provided. The assembly includes a planar structure comprising a first surface, a sanitization system and a divider. The sanitization system may include a housing having an interior volume with an opening on one side of the housing, wherein the housing is coupled to the planar structure. The sanitization system may also have a moveable sanitizing members, wherein the sanitizing members move in response to activation of the sanitization system. The divider may be coupled within the housing with a portion of the divider extending through the opening of the housing to extend away from the first surface of the planar structure or may be coupled adjacent the opening of the housing and extending away from the first surface of the planar structure. The sanitization system automatically sanitizes the divider with sanitization solution during movement of the sanitizing members after activation of the sanitization system.

### CROSS REFERENCE TO RELATED APPLICATION[S]

This application is a continuation-in-part of U.S. patent application entitled “RETRACTABLE SELF-SANITIZING DIVDER ASSEMBLY,” Ser. No. 17/201,861, filed Mar. 15, 2021, which claims priority to U.S. Provisional patent application entitled “RETRACTABLE SELF-SANITIZING DIVDER ASSEMBLY,” Ser. No. 63/022,419 filed May 8, 2020, the disclosures of which are hereby incorporated entirely herein by reference.

### BACKGROUND OF THE INVENTION

### Technical Field

This invention relates generally to a divider for use on a desk or other shared surfaces, and more particularly to a retractable self-sanitizing divider assembly that has a divider that is moveable between a retracted position and an extended position.

### State of the Art

Dividers are used for various purposes. Referring to FIG. 1, many workspaces, such as the desk of several computers, like at a computer lab in a university or an open workspace in a business lack dividers. Some solutions may be to provide to separate workspaces, dinner spaces or other types of spaces. These dividers are either formed as part of the surface structure or may be mounted to the structure. Each of these dividers suffer from the same issues. They each lack an ability to be removed without uninstalling and lack the ability to be easily sanitized or self-sanitized. The conventional way of cleaning or sanitizing these existing dividers is for a person to use cleaning products or the like in order to clean the surfaces. Additionally, the dividers are always present and there may be times when a divider is not needed or desired, and the conventional way to accomplish such is to uninstall it.

There is a need for an improved divider that can be moved between a retracted position and an extend position and reduces the time and effort for cleaning the divider.

### SUMMARY OF THE INVENTION

The present invention relates to a retractable self-sanitizing divider assembly with a divider that is moveable between a retracted position and an extended position.

An embodiment includes a self-sanitizing divider assembly comprising: a planar structure comprising a first surface; a sanitization system comprising: a housing comprising an interior volume with an opening on one side of the housing providing access to the interior volume of the housing, wherein the housing is coupled to the planar structure; and moveable sanitizing members, wherein the sanitizing members move in response to activation of the sanitization system; and a divider coupled within the housing with a portion of the divider extending through the opening of the housing to extend away from the first surface of the planar structure or coupled adjacent the opening of the housing and extending away from the first surface of the planar structure, wherein the sanitization system automatically sanitizes the divider with sanitization solution during movement of the sanitizing members after activation of the sanitization system.

An embodiment may include a retractable self-sanitizing divider assembly comprising: a planar structure comprising a first surface and a second surface; a housing comprising an interior volume with an opening on one side of the housing providing access to the interior volume of the housing, wherein the housing is coupled to the planar structure; a divider coupled within the housing moveable between a retracted position and an extended position, wherein the retracted position the divider is located within the housing and the in the extended position, a portion of the divider is extended through the opening of the housing to extend away from the first surface of the planar structure; and a sanitization system coupled to the housing wherein the sanitization system automatically sanitizes the divider during movement of the divider between the retracted position and the extended position.

In embodiments, the divider may be spring loaded. In other embodiments, the divider is a manually moved between the retracted position and the extended position. In yet further embodiments, the divider comprises an actuator that is electrically operated to move the divider between the retracted position and the extended position. The divider may be a planar substrate. The housing may be an open box shape. The sanitization system may comprise a flexible substrate. Sanitizing solution may be retained in the flexible substrate. The sanitizing solution may be located adjacent the opening of the housing. The flexible substrate may be coupled to a cassette. The cassette may be removeably coupled to the housing. The cassette may be removeably coupled to the divider. Sanitizing solution may be incorporated into a spray device sprayed on the flexible substrate. The assembly may be coupled between seats. The assembly may be coupled to an armrest between the seats. The seats may be shared transportation seats. The seats may be venue seats.

The foregoing and other features and advantages of the present invention will be apparent from the following more detailed description of the particular embodiments of the invention, as illustrated in the accompanying drawings.

### BRIEF DESCRIPTION OF THE DRAWINGS

A more complete understanding of the present invention may be derived by referring to the detailed description and claims when considered in connection with the Figures, wherein like reference numbers refer to similar items throughout the Figures, and:

FIG. 1 is a view of various prior art dividers;

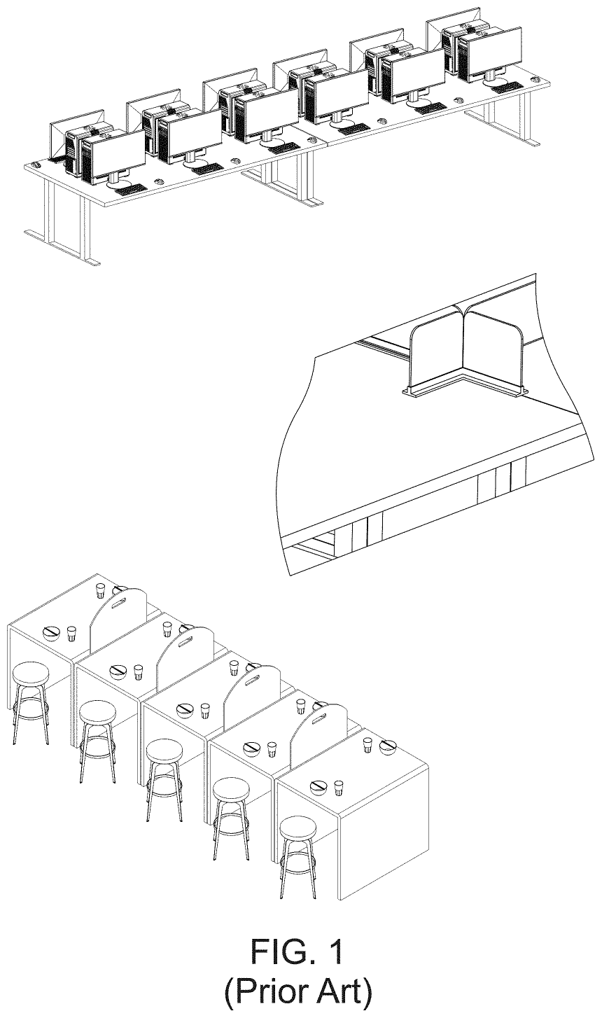

FIG. 2 is a perspective view of a table with a retractable self-sanitizing divider assembly coupled thereto with the divider in an extend position according to an embodiment;

FIG. 3 is a perspective view of a table with a retractable self-sanitizing divider assembly coupled thereto with the divider in a retracted position according to an embodiment;

FIG. 4 is a close-up partial view of a table with a retractable self-sanitizing divider assembly coupled thereto moving between a retraced position and an extended position according to an embodiment;

FIG. 5A is a section view of a table with a retractable self-sanitizing divider assembly coupled thereto moving between a retraced position and an extended position according to an embodiment;

FIG. 5B is another section view of a table with a retractable self-sanitizing divider assembly coupled thereto moving between a retraced position and an extended position according to an embodiment;

FIG. 5C is a section view of a table with a retractable self-sanitizing divider assembly coupled thereto moving between a retraced position and an extended position according to an embodiment;

FIG. 5D is another section view of a table with a retractable self-sanitizing divider assembly coupled thereto moving between a retraced position and an extended position with sanitizing solution sprayed onto a flexible substrate according to an embodiment;

FIG. 5E is another section view of a table with a retractable self-sanitizing divider assembly coupled thereto moving between a retraced position and an extended position with sanitizing solution sprayed onto the divider according to an embodiment

FIG. 6A is a view of a table with a retractable self-sanitizing seating and desk assemblies coupled thereto with the seating and table in a retracted position according to an embodiment;

FIG. 6B is a view of a table with a retractable self-sanitizing seating and desk assemblies coupled thereto with the seating and table in an extended position according to an embodiment;

FIG. 7A is a perspective view of a table with a retractable self-sanitizing divider assembly coupled thereto with the divider in an extend position according to an embodiment;

FIG. 7B is a perspective view of a table with a retractable self-sanitizing divider assembly coupled thereto with the divider in a retracted position according to an embodiment;

FIG. 7C is a perspective view of a person carrying a portable retractable self-sanitizing divider assembly according to an embodiment;

FIG. 8A is a view of seats with a retractable self-sanitizing divider assembly coupled thereto with the divider in an extend position according to an embodiment;

FIG. 8B is a view of seats with a retractable self-sanitizing divider assembly coupled thereto with the divider in a retracted position according to an embodiment;

FIG. 9A is a view of a self-order kiosk with a retractable self-sanitizing order assembly coupled thereto with the order device in an extend position according to an embodiment;

FIG. 9B is a view of a self-order kiosk with a retractable self-sanitizing order assembly coupled thereto with the order device in a retracted position according to an embodiment;

FIG. 10A is a perspective view of a table with a self-sanitizing divider assembly coupled thereto with a sanitizing member moving to sanitize the divider according to an embodiment;

FIG. 10B is a section view of a table with a self-sanitizing divider assembly coupled thereto with a sanitizing member moving to sanitize the divider according to an embodiment;

FIG. 10C is a section view of a table with a self-sanitizing divider assembly coupled thereto with a sanitizing member moving to sanitize the divider according to an embodiment;

FIG. 10D is a perspective view of a table with a self-sanitizing divider assembly coupled thereto with a sanitizing member moving to sanitize the divider according to an embodiment; and

FIG. 10E is an end view of a table with a self-sanitizing divider assembly coupled thereto with a sanitizing member moving to sanitize the divider according to an embodiment

### DETAILED DESCRIPTION OF EMBODIMENTS OF THE INVENTION

As discussed above, embodiments of the present invention relate to a retractable self-sanitizing divider assembly with a divider that is moveable between a retracted position and an extended position.

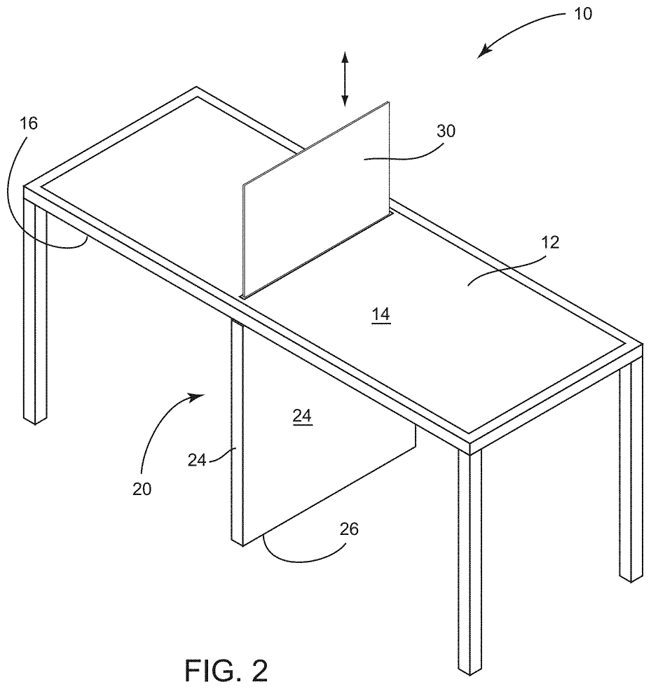

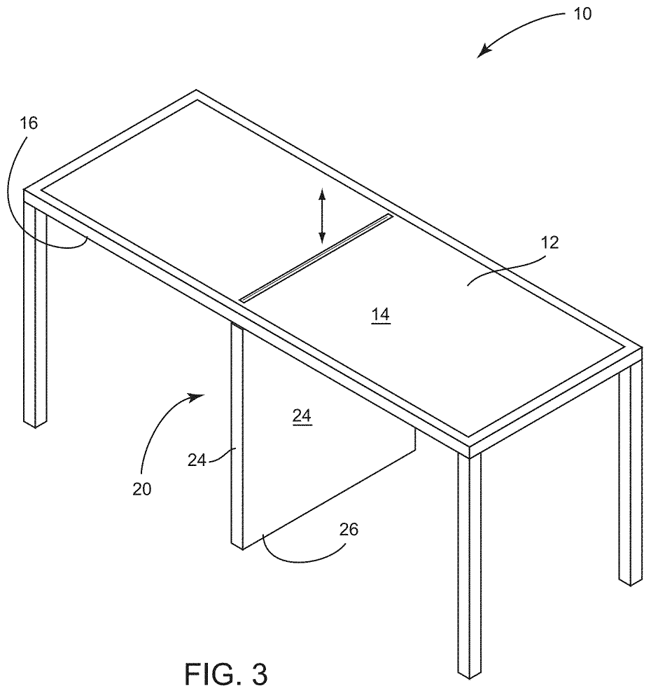

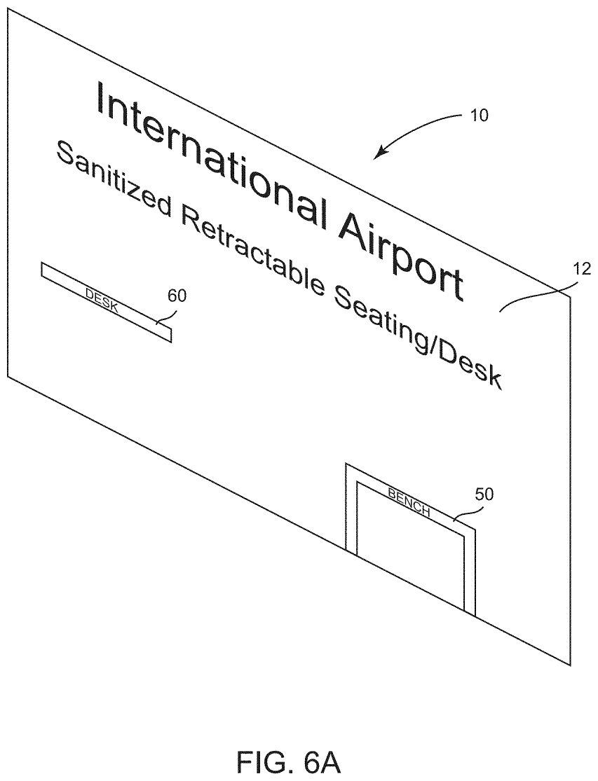

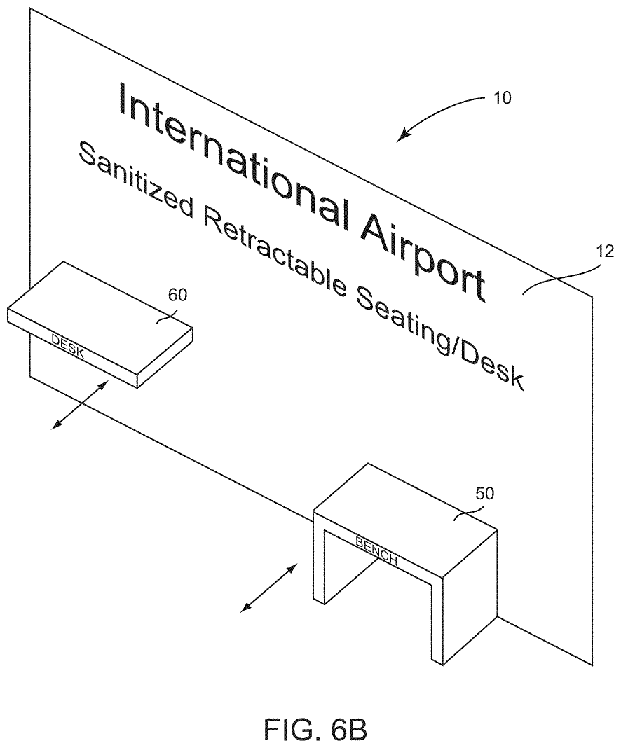

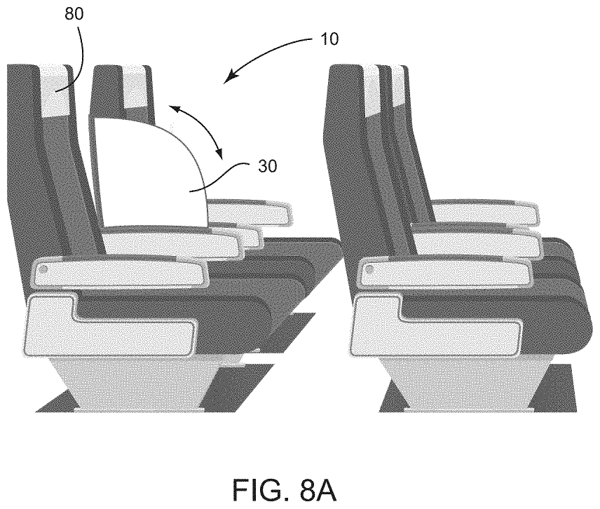

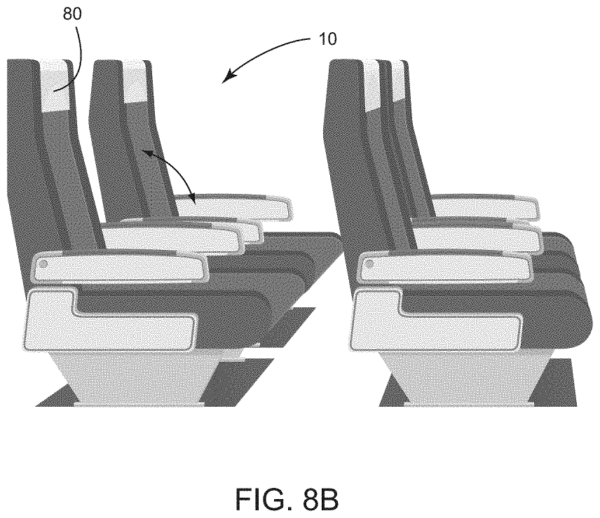

Referring to the drawings, FIGS. 2-5D depict an embodiment a retractable self-sanitizing divider assembly 10. The assembly 10 includes a planar structure 12, such as, but not limited to a desk to table structure (FIGS. 2-5D and 7A-7B), a wall (FIGS. 6A-6B), a chair (FIGS. 8A-8B) or the like. As shown in FIGS. 2-5D, the planar structure 12 is a table or desk surface. The planar structure 12 may include a first surface 14, a second surface 16, and in some embodiments may include an aperture 18 extending through the planar structure 12.

The assembly 10 may also include a housing 20 that includes an interior volume 22. In embodiments, the housing 20 may be a rectangular prism shape or any shape that corresponds to the shape of a divider 30. For example, and without limitation, the housing 20 may include four side members 24 that are coupled together to form a rectilinear shape and a bottom member 26 coupled to one end of each of the four side members 24 to form an open box structure, wherein the housing including an opening 28.

In some embodiments, the housing 20 may be coupled to the planar structure 12 with the housing extending away from the second surface 16 of the planar structure 12 with the opening 28 of the housing 20 aligned with the aperture 18 of the planar structure 12, as shown in FIGS. 2-5D.

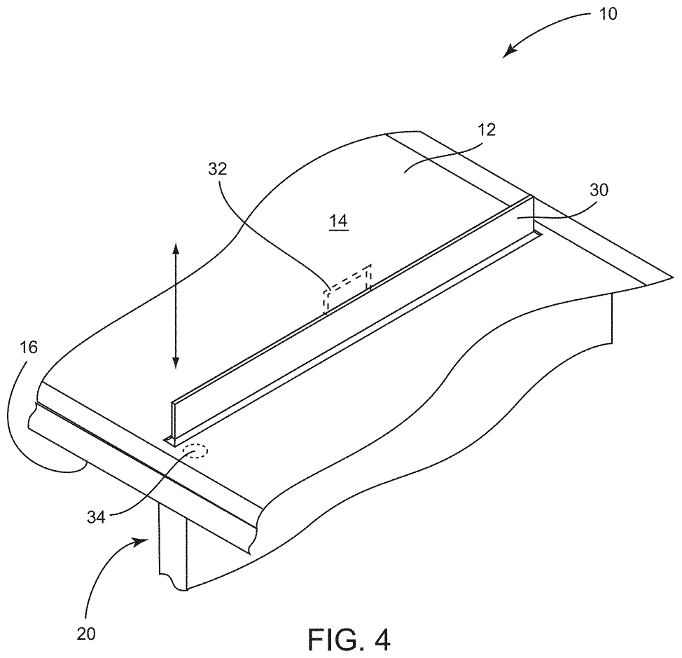

As depicted in FIGS. 2-5D, the assembly 10 may include a divider 30 that is moveable between a retracted position and an extended position. In the retracted position, the divider 30 is located within the housing 20 and the in the extended position, a portion of the divider 30 is extended through the opening 28 of the housing 20 and the aperture 18 of the planar structure 12 to extend away from the first surface 14 of the planar structure 12. The divider 30 in some embodiments may be planar substrate. Further, the divider 30 may be spring loaded with a locking device, wherein the spring biases the divider 30 toward the extended position and the locking device temporarily locks the divider 30 in the retracted position. In embodiments, the divider is a manually moved between the retracted position and the extended position, such as applying a force to the divider in order to engage the spring loaded device or to manually push or pull on a handle 32 (see optional handle 32 in FIG. 4). In yet further embodiments, the divider 30 comprises an actuator that is electrically operated to move the divider 30 between the retracted position and the extended position, such as by using a button 34 shown in FIG. 4 or by remotely activating the electric actuator.

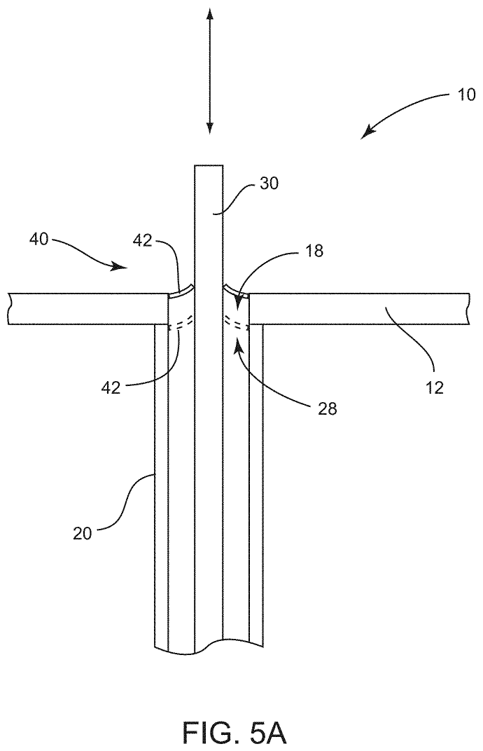

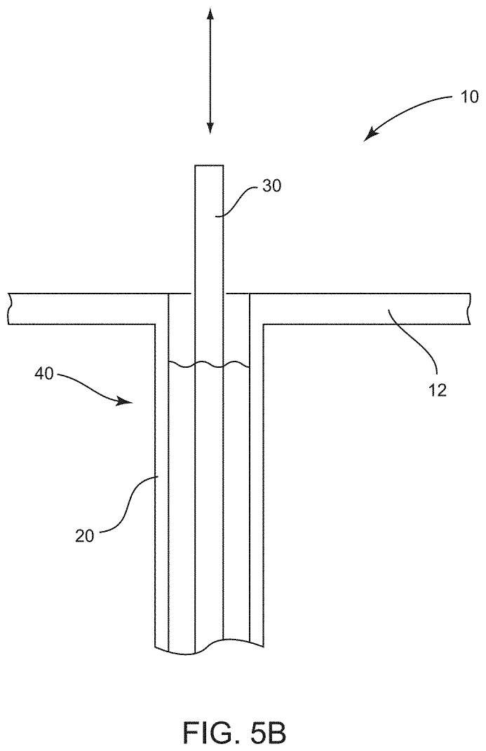

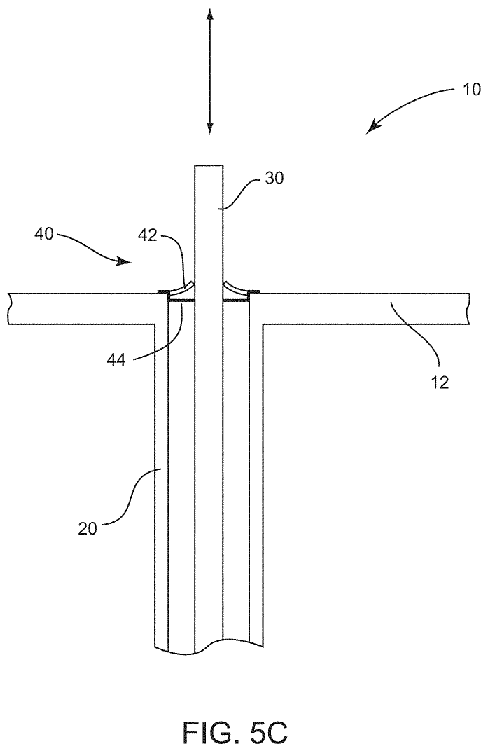

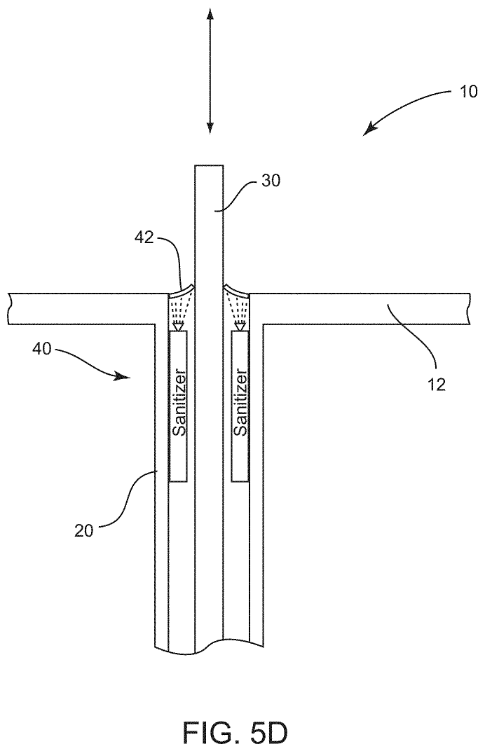

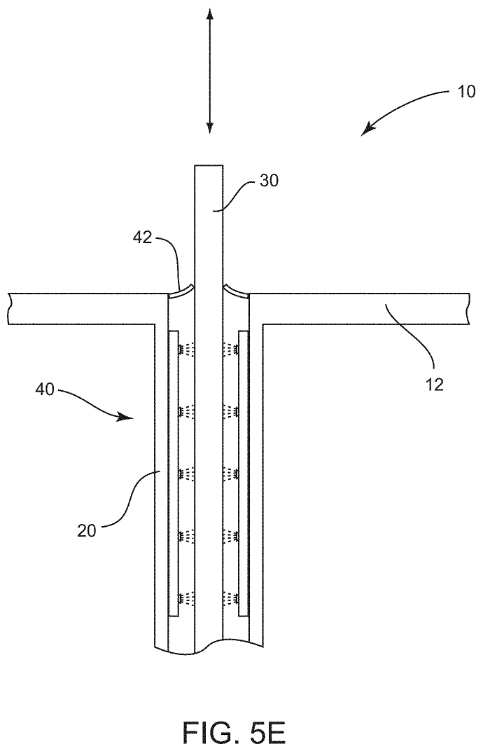

As shown in FIGS. 2-5E, the assembly 10 may also include a sanitization system 40. The sanitization system 40 may include a flexible substrate 42 (see FIGS. 4, 5A, 5C, 5D and 5E). In embodiments, the sanitizing solution may be retained within the flexible substrate 42 and located in various positions adjacent the opening of the housing, as shown in FIGS. 4 and 5A. In another embodiment, the flexible substrate 42 may be coupled to a cassette 44, wherein the cassette 44 is removably coupled to the housing 20 or the planar structure 12 and sanitizing solution may be retained within the flexible substrate 42, as shown in FIG. 5C. The cassette 44 with the flexible substrate 42 may be replaced by removing and installing another in its place, wherein the replacement of the cassette 44 occurs when the flexible substrate 42 no longer retains sanitizing solution. In FIG. 5D, embodiments may include the sanitizing solution incorporated into a spray device 46 that sprays the flexible substrate 42 with sanitizing solution as the divider 30 moves between the retracted position and the extended position. In FIG. 5E, embodiments may include the sanitizing solution incorporated into a spray device 46 that sprays the divider 30 with sanitizing solution as it moves between the retracted position and the extended position. Another embodiment depicted in FIG. 5B, includes a sanitizing solution 44 retained within the housing 20, such that the divider 30 is immersed in the sanitizing solution 44 for sanitization purposes. In this embodiment with the sanitization solution retained within the housing 20, a flexible substrate 42 may be utilized and may operate as a squeegee to wipe off the sanitization solution as the divider 30 moves from the retracted position to the extended position.

Generally, suitable sanitizing solutions comprise disinfectants approved for use against COVID-19 on surfaces by the Environmental Protection Agency (EPA), such as hydrogen peroxide and the like. EPA List N: Disinfectants for Use Against SARS-CoV-2 lists disinfectants approved by the EPA and is hereby incorporated entirely herein by reference.

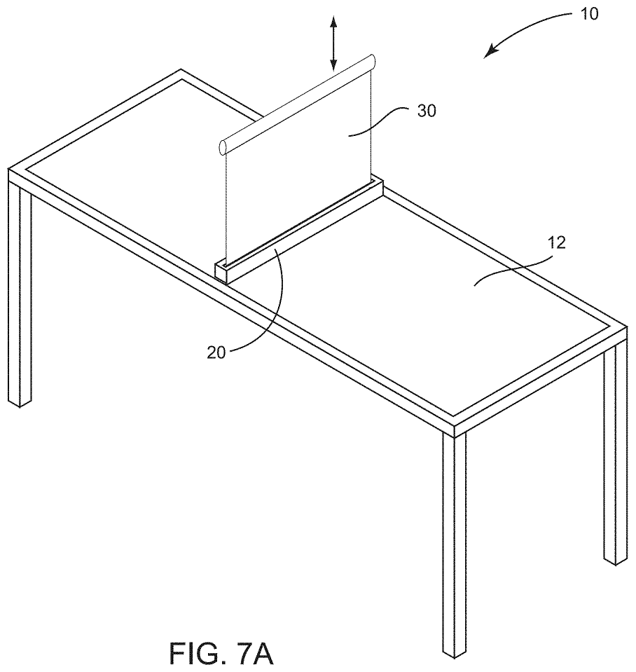

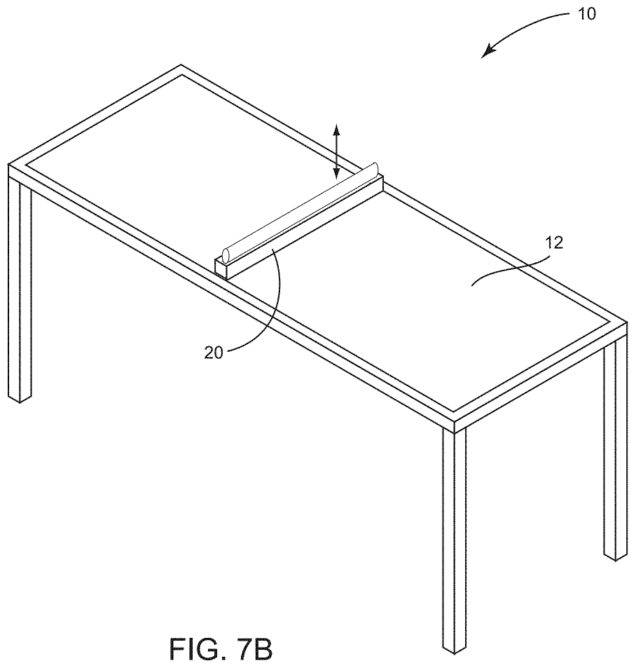

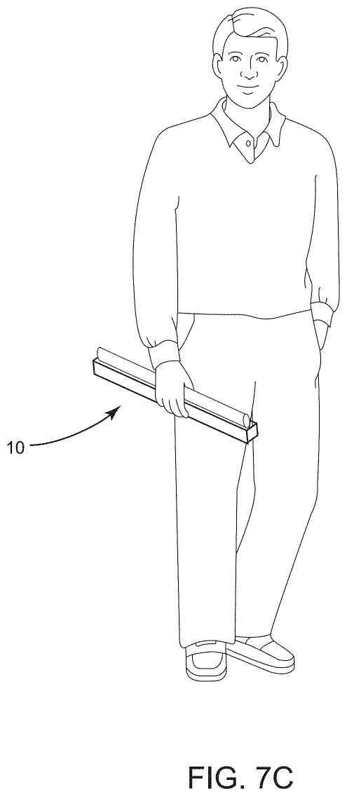

In other embodiments, as shown in FIGS. 7A-7C, the assembly 10 may include a housing 20 with a divider 30 that may be coupled to the first surface of the planar structure 12, and the assembly 10 may be portable. The housing 20 comprises an opening with the divider 30 that extends therethrough. The divider 30 may be formed of a material that allows it to roll onto a reel or other cylindrical member within the housing 20. In the retracted position, the divider 30 is rolled onto the cylindrical member within the housing 20 and the in the extended position, the divider 30 is extended through the opening of the housing 20 and retained in a vertical position. The divider 30 may be formed of a material that allows for this functionality or may include a structure that operates to hold flexible material in the vertical position. In embodiments, the assembly 10 is portable and a person may transport the assembly 10 by carrying it with him or her to the locations that the individual may wish to utilize a divider.

In some embodiments, the assembly 10 may be utilized for other structures beyond a divider 30. For example, as depicted in FIGS. 6A and 6B, the assembly 10 may operate for seating structures 50 and desk structures 60 that are moveable between retracted positions and extend positions from the planar structure 12 that is a wall at an airport for passengers waiting to board a plane. The operation is the same for these structures as it is for the divider, wherein the seating structures 50 and desk structures 60 may be moved between a retracted position (FIG. 6A) and an extended position (FIG. 6B), including the sanitization of the seating structures 50 and desk structures 60 when moving between the extended position and the retracted position.

Further still, as shown in FIGS. 8A-8B, the assembly 10 may be coupled to arm rests or otherwise between seats, such as seats 80 on airlines, buses, trains or other shared transportation, and further may be used between other venue seats, like theater seats, sporting event seats or the like. In these embodiments, the divider 30 may be a flexible material and the housing 20 may include a hinged member 24 hingedly coupled to the housing 20 on one end. The hinged member 24 may be coupled to an end of the divider 30 and the opposing end of the divider may be coupled to the housing 20. Rotation of the hinged member 24 away from the housing 20 results in moving the divider from the retracted position to the extended position, wherein the divider is a partial circular shape. The rotation of the hinged member 24 toward the housing 20 results in the divider being moved from the extended position to the retracted position with the divider 30 within the housing 20. These embodiments may be incorporated into the arm rests of the seats 80 or may be after market products coupled to the seats 80.

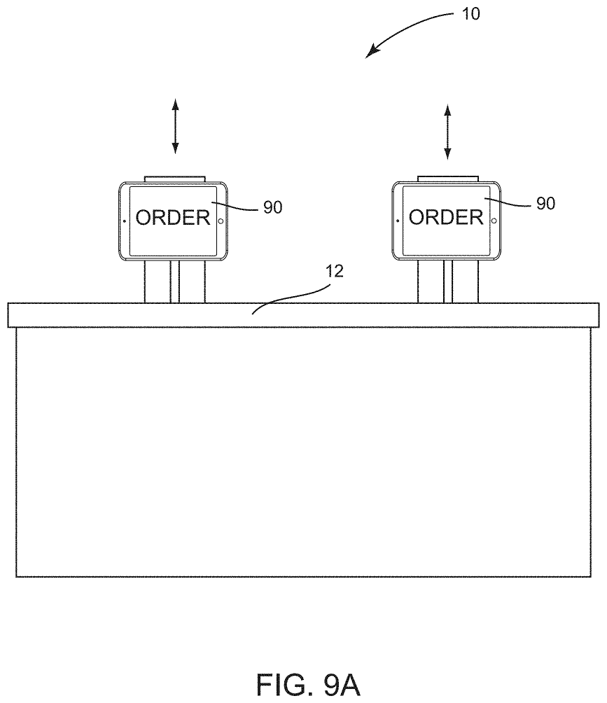

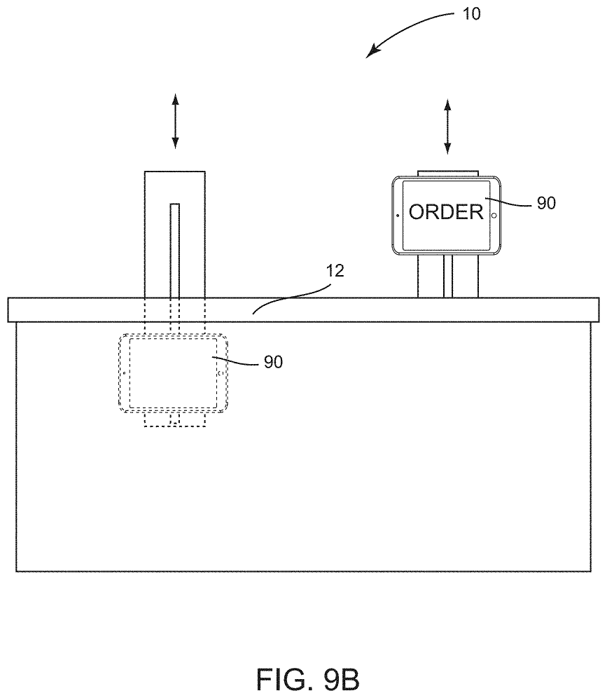

In additional embodiments, the assembly 10 may be utilized for other purposes and devices beyond a divider 30. For example, as depicted in FIGS. 9A and 9B, the assembly 10 may operate for order devices 90 that are moveable between retracted positions and extend positions from the planar structure 12 that is a flat surface of an order kiosk 92. The operation is the same for these order devices 90 as it is for the divider, wherein the order devices 90 may be moved between a retracted position (FIG. 9B) and an extended position (FIG. 9A), including the sanitization of the order device 90 when moving between the extended position and the retracted position. In embodiments, once an order is completed, the assembly 10 may automatically operate to move the device 90 from the extended position to the retracted position to be sanitized, and then the device may automatically be moved from the retracted position to the extended position to receive another order.

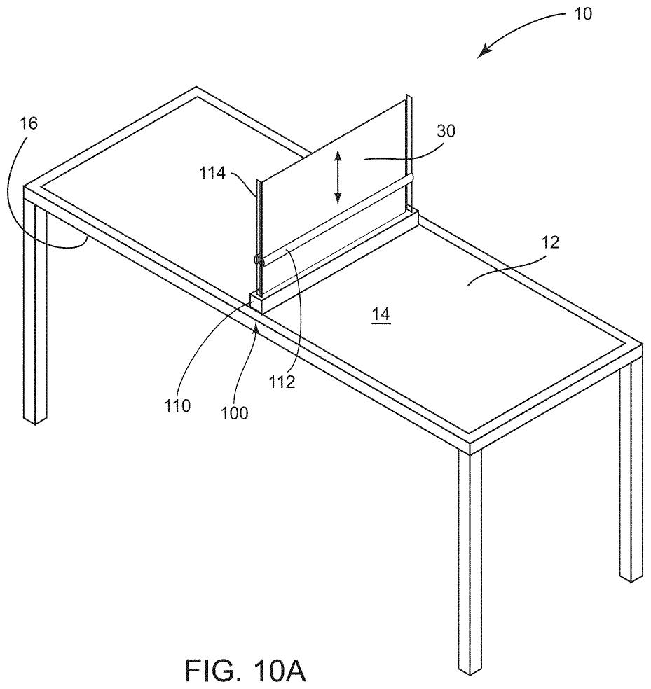

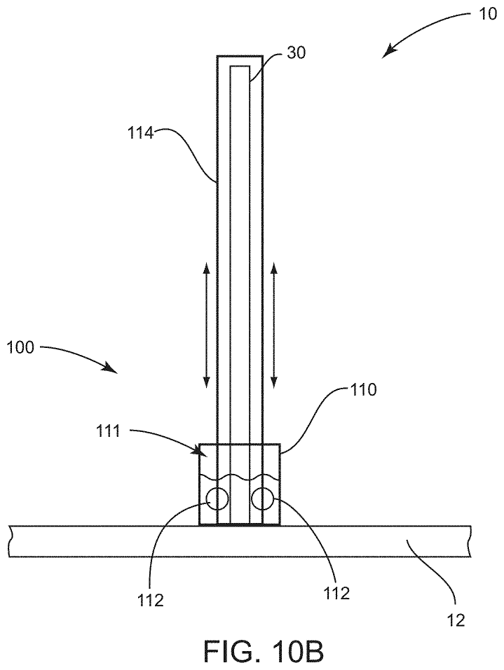

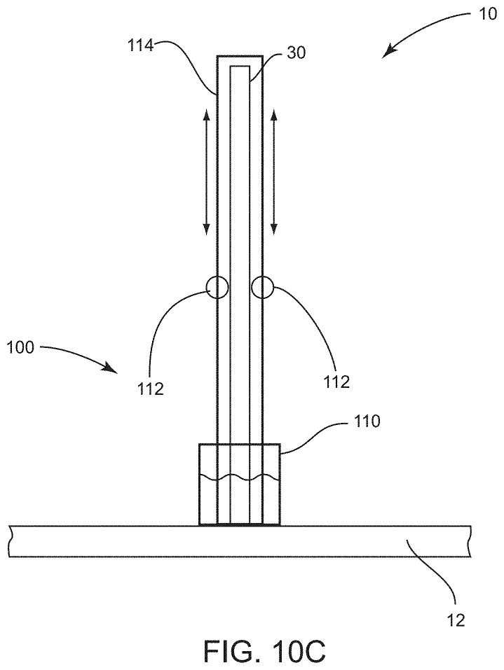

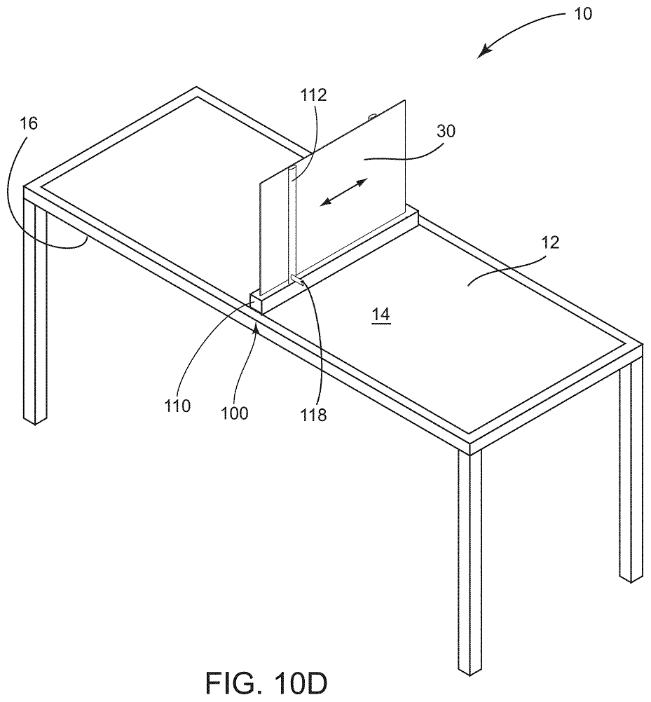

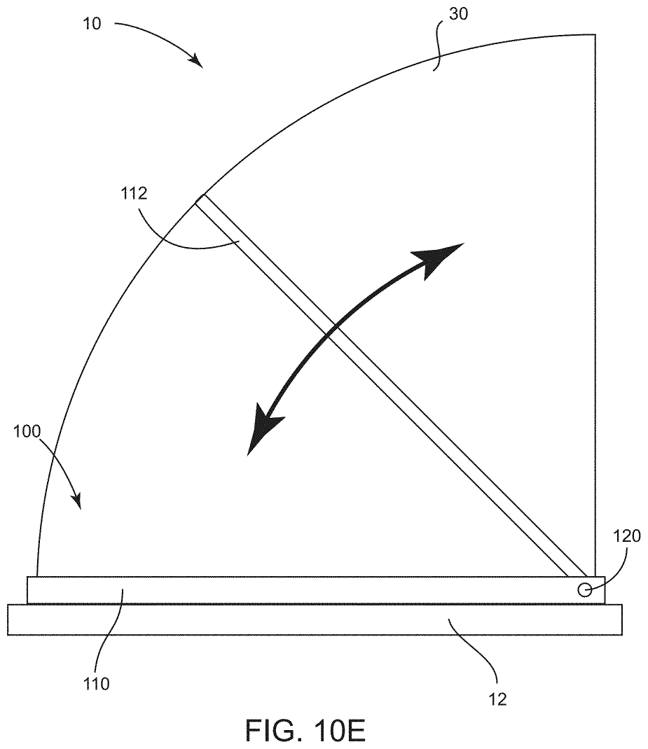

Another embodiment is shown in FIGS. 10A-10E comprising a self-sanitizing divider assembly 10 that includes a sanitization system 100 and a divider 30. The assembly 10 may be coupled to a planar structure 12, such as, but not limited to a desk to table structure, a wall, a chair or the like. As shown in FIGS. 10A-10E, the planar structure 12 is a table or desk surface, but this is only for exemplary purposes of this disclosure. The planar structure 12 may include a first surface 14, a second surface 16.

The system 110 may also include a housing 110 that includes an interior volume 111. In embodiments, the housing 110 may be a rectangular prism shape or any shape that corresponds to the shape of a divider 30. For example, and without limitation, the housing 110 may include four side members that are coupled together to form a rectilinear shape and a bottom member coupled to one end of each of the four side members to form an open box structure, wherein the housing includes an opening. The housing 110 may be coupled to the planar structure, such as securing it with bolts, adhesive, resting it on the surface and so forth.

The assembly 10 may include sanitizing members 112 operatively coupled on opposing sides of the divider 30. As shown in FIGS. 10A-10C, the sanitizing members 12 move vertically to sanitize the divider 30. In this embodiment, a sanitizing solution may be retained within the housing 110, such that the sanitizing members 112 may be immersed in the sanitizing solution as shown in FIG. 10B in order for the sanitizing members to absorb some of the sanitizing solution. Then during operation, an electro-mechanical device may include a vertical track 114, as shown in FIG. 10C that the sanitizing members 112 travel along, such as by a pulley and chain, an actuator, a geared connection and so forth, that moves the sanitizing members vertically up to apply sanitizing solution onto the divider 30 to sanitize the divider 30 and then move the sanitizing members 112 back into the housing 110.

In another embodiment shown in FIG. 10D, the sanitizing members 112 may be positioned vertically and the mechanism moves the sanitizing members 112 horizontally along the surfaces of the divider 30 to apply sanitizing solution to the divider 30. The sanitizing members may include a material that can wick the solution from within the housing 110 to ensure that solution is applied to the entire surface of the divider 30 by each sanitizing member 112.

In yet another embodiment, the sanitizing member 112 may be a rotatable member that can rotate to clean the divider 30, as shown in FIG. 10E. The sanitizing member 112 may be located within the sanitization fluid within the housing 110 and then upon activation, the sanitizing members 112 rotate out of the housing 110 and apply sanitization solution to the surfaces of the divider 30, thereby sanitizing the divider 30. In such embodiments, the divider 30 may be an arcuate divider in order to ensure the sanitizing members 30 sanitize the entire surface of the divider 30.

While is shown in FIGS. 10A-10E that the sanitization fluid is held within the housing 110 and the sanitizing members 112 are immersed within the sanitization solution, other embodiments are contemplated. For example, a spray device, such as that shown in FIG. 5D may be employed within the housing to spray sanitization solution directly on the sanitizing member 112. Additionally, the sanitizing member 12 may include a sanitizing substrate that is replaceable, wherein the sanitizing substrate is preloaded with sanitization solution and is configured to last for a certain period of time before the sanitization solution preloaded on the sanitizing substrate is dry and then the sanitizing substrate may be replaced with another that is preloaded with sanitization solution.

It will be understood that the sanitization system 100 may include a power source, such as a plug to a power source or batteries. The system 100 may be activated by a push of a button (such as button 120 in FIG. 10E) that operates the electro-mechanical system to move the sanitizing members 112 over the surfaces of the divider 30. Some embodiments may include a single button that is pushed to start and stop, and other embodiments may include separate buttons for start and stop and further still, other embodiments may include a single button that starts or activated the system 100 and then the system automatically stops after the sanitizing members 112 move for a complete cycle of back and forth over the surfaces of the divider 30. Additionally, the system 100 may be automated, wherein the system 100 is activated automatically based on motion, light, time lapsing or the like. Further still, the system may allow a user to manual operate the sanitization system 100, wherein the user manually moves the sanitizing members 112 to sanitize the divider 30. For example, as shown in FIG. 10D, a control member 118 may be coupled to the sanitizing members 112, wherein the user may engage the control member 118 with he or her hand and move the sanitizing members 112 through a sanitizing cycle. In at least these ways, the system 100 may be activated by the means of automated activation, electrical activation by push button or manual activation.

Embodiments may be available on or through the internet, such as through domain names reserved and owned by Applicant that include auto-wipe.com, sani-swipe.com, wipeoutgerm.com, wipeout-virus.com, dipandclean.com, friendlydivider.com, pushtoclean.com, cleanpopup.com, magicdivider.com, and the like.

Accordingly, the components defining any retractable self-sanitizing divider assembly or self-sanitizing divider assembly may be formed of any of many different types of materials or combinations thereof that can readily be formed into shaped objects provided that the components selected are consistent with the intended operation of a retractable self-sanitizing divider assembly or a self-sanitizing divider assembly. For example, the components may be formed of: rubbers (synthetic and/or natural) and/or other like materials; glasses (such as fiberglass) carbon-fiber, aramid-fiber, any combination thereof, and/or other like materials; polymers such as thermoplastics (such as ABS, Fluoropolymers, Polyacetal, Polyamide; Polycarbonate, Polyethylene, Polysulfone, and/or the like), thermosets (such as Epoxy, Phenolic Resin, Polyimide, Polyurethane, Silicone, and/or the like), any combination thereof, and/or other like materials; composites and/or other like materials; metals, such as zinc, magnesium, titanium, copper, iron, steel, carbon steel, alloy steel, tool steel, stainless steel, aluminum, any combination thereof, and/or other like materials; alloys, such as aluminum alloy, titanium alloy, magnesium alloy, copper alloy, any combination thereof, and/or other like materials; any other suitable material; and/or any combination thereof.

Furthermore, the components defining any retractable self-sanitizing divider assembly or self-sanitizing divider assembly may be purchased pre-manufactured or manufactured separately and then assembled together. However, any or all of the components may be manufactured simultaneously and integrally joined with one another. Manufacture of these components separately or simultaneously may involve extrusion, pultrusion, vacuum forming, injection molding, blow molding, resin transfer molding, casting, forging, cold rolling, milling, drilling, reaming, turning, grinding, stamping, cutting, bending, welding, soldering, hardening, riveting, punching, plating, and/or the like. If any of the components are manufactured separately, they may then be coupled with one another in any manner, such as with adhesive, a weld, a fastener (e.g. a bolt, a nut, a screw, a nail, a rivet, a pin, and/or the like), wiring, any combination thereof, and/or the like for example, depending on, among other considerations, the particular material forming the components. Other possible steps might include sand blasting, polishing, powder coating, zinc plating, anodizing, hard anodizing, and/or painting the components for example.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above without departing from the spirit and scope of the forthcoming claims.

1. A self-sanitizing divider assembly comprising:
a planar structure comprising a first surface;
a sanitization system comprising:
a housing comprising an interior volume with an opening on one side of the housing providing access to the interior volume of the housing, wherein the housing is coupled to the planar structure; and
moveable sanitizing members, wherein the sanitizing members move in response to activation of the sanitization system; and
a divider coupled within the housing with a portion of the divider extending through the opening of the housing to extend away from the first surface of the planar structure or coupled adjacent the opening of the housing and extending away from the first surface of the planar structure, wherein the sanitization system automatically sanitizes the divider with sanitization solution during movement of the sanitizing members after activation of the sanitization system.

2. The assembly of claim 1, wherein the sanitization system comprises sanitization solution within the housing, wherein the sanitizing members are at least partially immersed within the sanitization solution prior to activation of the sanitization system.

3. The assembly of claim 1, wherein the sanitization system comprises spray devices configured to spray sanitization solution onto the sanitizing members prior to the sanitizing members moving after activation of the sanitization system.

4. The assembly of claim 1, wherein the sanitizing members comprise a sanitizing substrate preloaded with sanitization solution.

5. The assembly of claim 4, wherein the sanitizing substrate is replaceable when the sanitization solution preloaded on the sanitization substrate is evaporated.

6. The assembly of claim 1, wherein the planar structure is a table or desk.

7. The assembly of claim 1, wherein the planar structure is a counter.

8. The assembly of claim 1, wherein the planer structure is an armrest of a seat.
